SEQUENCE LISTING (20 sequences)

SEQ ID NO: 1 - DNA, 1801 bp, Homo sapiens
  source 1..1801: mol_type = other DNA
gcaaggcatagagacaacatagagctaagtaaagccagtggaaatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggccatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagacaagtgactgtatctgtgtagactatttgcttatttaataaagacgatttgtcagttgtttt

SEQ ID NO: 2 - DNA, 1440 bp, Mus musculus
  source 1..1440: mol_type = other DNA
atgaaaatgaagggtcttccggtcctgctgtggctgtgtgtggttgtgtgctcatcctacccattgcatgacagtgcaagggatgatgatgctggtatggagcttctgcagaaatacctagaaaactactatggccttgcaaaagatgtgaagcaatttattaagaaaaaggacagtagtcttattgtcaaaaaaattcaagaaatgcagaagttcctcgggttggagatgacagggaagctggactccaacactatggagctgatgcataagcccaggtgtggtgttcctgatgttggtggcttcagtaccttcccaggttcgccaaaatggaggaaatcccacatcacctacaggattgtgaattatacaccggatttgccaagacagagtgtggattctgccattgaaaaagctttgaaggtctgggaggaggtgaccccactcactttctccaggatctctgaaggagaggctgacataatgatctcctttgcagttggagaacatggagactttgtcccttttgatgggcctggaacagtcttggctcatgcctatgcacctggaccagggattaatggagatgctcactttgacgatgatgaacgatggacagaggatgtcactggtaccaacctattcctggttgctgctcatgaacttggccactccctgggactctaccactcagccaaggctgaagctctgatgtacccagtctacaagtcctccacagacttgtcccgtttccatctctctcaagatgatgtagatggtattcagtccctctatggaactcccacagcatcccctgatgtcctcgtggtacccaccaagtctaactctctggaacctgagacatcaccaatgtgcagctctactttgttctttgatgcagtcagcaccctccggggagaagtcctgttttttaaagacaggcacttttggcgcaaatctctcaggactcctgagcctgaattttatttgatctcttcattttggccatctcttccatccaacatggatgctgcatatgaggttactaacagagacactgttttcatttttaaaggaaatcagttctgggctatacgagggcacgaggagctagcaggttatcctaaaagcattcacaccctgggtctccctgcaaccgtgaagaagatcgatgctgccatttctaataaagagaaaaggaagacctacttctttgtagaggacaaatactggaggtttgatgagaagaaacaatccatggagccaggatttcccaggaagatagctgaggactttccaggtgttgactcaagggtggatgctgtctttgaagcatttgggtttctctacttcttcagtggatcttcgcagttggaatttgacccaaatgccaaaaaagtgacccacatattgaagagcaatagctggtttaattgttaa

SEQ ID NO: 3 - DNA, 1815 bp, Homo sapiens
  source 1..1815: mol_type = other DNA
atgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtag

SEQ ID NO: 4 - DNA, 1815 bp, Mus musculus
  source 1..1815: mol_type = other DNA
atgctcttccgagctgtgctgctctgcgctgccctggggctcagccaggcagcaaatccttgctgttccaatccatgtcaaaaccgtggggaatgtatgagcacaggatttgaccagtataagtgtgactgtacccggactggattctatggtgaaaactgtactacacctgaatttctgacaagaatcaaattactgctgaagcccaccccaaacacagtgcactacatcctgacccacttcaagggagtctggaacattgtgaacaacatccccttcctgcgaagtttaatcatgaaatatgtgctgacatccagatcatatttgattgacagtccacctacttacaatgtgcactatggttacaaaagctgggaagccttctccaacctctcctactacaccagggcccttcctcccgtagcagatgactgcccaactcccatgggtgtgaagggaaataaggagcttcctgattcaaaagaagtgctggaaaaggttcttctacggagagagttcatccctgacccccaaggctcaaatatgatgtttgcattctttgcccagcacttcacccatcagtttttcaagacagatcataagcgaggacctgggttcacccgaggactgggccatggagtggacttaaatcacatttatggtgaaactctggacagacaacataaactgcgccttttcaaggatggaaaattgaaatatcaggtcattggtggagaggtgtatccccccacagtcaaagacactcaggtagagatgatctaccctcctcacatccctgagaacctgcagtttgctgtggggcaggaagtctttggtctggtgcctggtctgatgatgtatgccaccatctggcttcgggagcacaacagagtgtgcgacatactcaagcaggagcatcctgagtggggtgatgagcaactattccaaaccagcagactcatactcataggagagactatcaagatagtgatcgaagactacgtgcaacacctgagcggttaccacttcaaactcaagtttgacccagagctccttttcaaccagcagttccagtatcagaaccgcattgcctctgaattcaacacactctatcactggcaccccctgctgcccgacaccttcaacattgaagaccaggagtacagcttcaaacagtttctctacaacaactccatcctcctggaacatggactcactcagtttgttgagtcattcaccagacagattgctggccgggttgctgggggaagaaatgtgccaattgctgtacaagcagtggcaaaggcctccattgaccagagcagagagatgaaataccagtctctcaatgagtaccgcaaacgcttctccctgaagccgtacacatcatttgaagaacttacaggagagaaggaaatggctgcagaattgaaagccctctacagtgacatcgatgtcatggaactgtaccctgccctgctggtggaaaaacctcgtccagatgctatctttggggagaccatggtagagcttggagcaccattctccttgaaaggacttatgggaaatcccatctgttctcctcaatactggaagccgagcacctttggaggcgaagtgggttttaagatcatcaatactgcctcaattcagtctctcatctgcaataatgtgaaggggtgtcccttcacttctttcaatgtgcaagatccacagcctaccaaaacagccaccatcaatgcaagtgcctcccactccagactagatgacattaaccctacagtactaatcaaaaggcgttcaactgagctgtaa

SEQ ID NO: 5 - DNA, 1425 bp, Homo sapiens
  source 1..1425: mol_type = other DNA
atgactctggcctacataccccacagtgccttgcggcgcaggccgctcccggatgtgtgcctggcgccggaagagaagacggcccccctctctcggcccggccatcttgtgggaagagctgaagcaggcgctcttggctcggcgcggcccgctgcaatccgtggaggaacgcgccgccgagccaccatcatgcctgggcacttacaggaaggcttcggctgcgtggtcaccaaccgattcgaccagttatttgacgacgaatcggaccccttcgaggtgctgaaggcagcagagaacaagaaaaaagaagccggcgggggcggcgttgggggccctggggccaagagcgcagctcaggccgcggcccagaccaactccaacgcggcaggcaaacagctgcgcaaggagtcccagaaagaccgcaagaacccgctgccccccagcgttggcgtggttgacaagaaagaggagacgcagccgcccgtggcgcttaagaaagaaggaataagacgagttggaagaagacctgatcaacaacttcagggtgaagggaaaataattgatagaagaccagaaaggcgaccacctcgtgaacgaagattcgaaaagccacttgaagaaaagggtgaaggaggcgaattttcagttgatagaccgattattgaccgacctattcgaggtcgtggtggtcttggaagaggtcgagggggccgtggacgtggaatgggccgaggagatggatttgattctcgtggcaaacgtgaatttgataggcatagtggaagtgatagatcttttgttagttctttttcacattacagtggcctgaagcacgaggacaaacgtggaggtagcggatctcacaactggggaactgtcaaagacgaattaacagagtcccccaaatacattcagaaacaaatatcttataattacagtgacttggatcaatcaaatgtgactgaggaaacacctgaaggtgaagaacatcatccagtggcagacactgaaaataaggagaatgaagttgaagaggtaaaagaggagggtccaaaagagatgactttggatgagtggaaggctattcaaaataaggaccgggcaaaagtagaatttaatatccgaaaaccaaatgaaggtgctgatgggcagtggaagaagggatttgttcttcataaatcaaagagtgaagaggctcatgctgaagattcggttatggaccatcatttccggaagccagcaaatgatataacgtctcagctggagatcaattttggagaccttggccgcccaggacgtggcggcaggggaggacgaggtggacgtgggcgtggtgggcgcccaaaccgtggcagcaggaccgacaagtcaagtgcttctgctcctgatgtggatgacccagaggcattcccagctctggcttaa

SEQ ID NO: 6 - DNA, 1179 bp, Mus musculus
  source 1..1179: mol_type = other DNA
atgcctgggcacctacaggaaggcttcggctgcgtggtcaccaaccgattcgaccagctatttgacgacgaatcggaccctttcgaggtactgaaggcagcagagaacaagaaaaaagaagccggcgggggcggcgttgggggccccggggccaagagcgcggctcaggccgcggcccagaccaactccaacgcggcgggcaaacagttgcgtaaagagtcccagaaagaccgcaagaacccgctgccccccagcgtcggcgtggccgacaaaaaggaggagacgcagccgccggtggcgcttaagaaagaaggaataaggcgagttggaagaagacccgatcaacaactacagggtgatgggaaactaattgataggagagcagaaaggcgaccaccccgtgaaagaagatttgaaaagccacttgaagaaaaaggtgaaggaggtgaattttcagttgatagaccgattatcgaacggcctatccgaggccgaggtggtcttggaaggggtcgaggaggccgtggacgtggaatgggccgaggcgatggatttgattctcgtggcaagcgtgaatttgataggcatagtggaagtgatagatcttctttttcacattacagtggcctgaagcatgaggacaaacgcggaggtagcggctctcacaactggggaactgtcaaagatgaattaactgatttggatcaatcaaatgtgactgaggaaacacctgaaggtgaagagcaccctgtggcagatactgaaaataaggagaacgaagttgaagaggttaaggaagagggtccaaaagagatgactctggatgagtggaaagctattcaaaataaagaccgagcaaaagtagaatttaatatccgaaaaccaaatgaaggcgccgatggacaatggaaaaagggatttgttctgcataaatcaaaaagtgaagaggctcatgctgaagattcagttatggaccatcatttccggaagccagcaaatgatataacatctcaactggagatcaattttggagacttaggccgcccaggacgtggtggcagaggaggacgtggtgggcgtgggcgtggtggacgtccaaaccgtggcagcaggactgataagtcaagtgcttctgctcctgatgtagatgacccagaggccttcccagctctggcctaa

SEQ ID NO: 7 - DNA, 1209 bp, Homo sapiens
  source 1..1209: mol_type = other DNA
atgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggacgcgaacgtggttttctcaccctatggggtggcctcggtgttggccatgctccagctgacaacaggagtagaaacccagcagcagattcaagcagctatgggattcaagattgatgacaagggcatggcccccgccctccggcatctgtacaaggagctcatggggccatggaacaaggacgagatcagcaccacagacgcgatcttcgtccagcgggatctgaagctggtccagggcttcatgccccacttcttcaagctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccacgtcatgagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctga

SEQ ID NO: 8 - DNA, 1248 bp, Mus musculus
  source 1..1248: mol_type = other DNA
atggaagaactttccatggcaaacaccatgtttgccctcaatctccttaagcagatagaaaaatcaaactctacccagaacatctttatctctccatggagcatctcatcaacattggccatagttctcctcggtgctgggggtaacactgaacagcagatggccaaagtgctgcagtttaatgaaattggcagttatggtatcaccacaagaaacccagagaacttcagtggctgtgatttcgcacaacagatacagaaggaaaattatcctagtgctattttacaggcacaagcaggagataaaatccattcagccttctcctctctcagctcaacaatcaacacaccacagggggattatttgttagaaagtgcaaacaagctgtttggagagaagtctgcaagattcaaagaagaatacatccaactctctaagaaatattactcaacagaaccagaagcagtggacttccttgaatgtgctgaagaagctagggaaaagattaattcttgggtcaagactcaaaccaaaggtgaaatcccaaacctgctacccgaaggttctgtagatgaagacaccaagatggtgctggtgaatgctgtctacttcaaaggaaagtggaaaactccatttgagaagaaacttaatgggctttatcctttccgtgtgaactcgcatgagagcatacctgtccagatgatgttcctccatgcaaagctgaacattggatacataaaggacctgaagactcagatcctagaacttccgcatactggaaacatcagcatgctcctgttgcttcccgatgagattgaggacgcatccactggcttggaattgctggaaagtgaaataaactttgccaacttcaacaagtggatcagcaaagacacactggatgaagatgatgttgtggtctacattcccaagttcaaactggcacaaagctacgaactcaagtccattcttcaaagcatgggcatggaggatgccttcaacaagggcaaggccaacttctcaggaatgtctgagaggaatgacctttttctttctgaggtgttccatcaagccagcgtggatgtcaccgaggagggcactgtggcagctggtgggactggggcagttatgacaggaagaactggccatggtggcccacagtttgtggccgatcatccctttcttttctttatcatggacaaaattacccacacgatactatttgttggtagattctcctcaccctaa

SEQ ID NO: 9 - DNA, 1296 bp, Homo sapiens
  source 1..1296: mol_type = other DNA
atgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcattgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctga

SEQ ID NO: 10 - DNA, 1302 bp, Mus musculus
  source 1..1302: mol_type = other DNA
atgaaagtctggctggcgagcctgttcctctgcgccttggtggtgaaaaactctgaaggtggcagtgtacttggagctcctgatgaatcaaactgtggctgtcagaacggaggtgtatgcgtgtcctacaagtacttctccagaattcgccgatgcagctgcccaaggaaattccagggggagcactgtgagatagatgcatcaaaaacctgctatcatggaaatggtgactcttaccgaggaaaggccaacactgataccaaaggtcggccctgcctggcctggaatgcgcctgctgtccttcagaaaccctacaatgcccacagacctgatgctattagcctaggcctggggaaacacaattactgcaggaaccctgacaaccagaagcgaccctggtgctatgtgcagattggcctaaggcagtttgtccaagaatgcatggtgcatgactgctctcttagcaaaaagccttcttcgtctgtagaccaacaaggcttccagtgtggccagaaggctctaaggccccgctttaagattgttgggggagaattcactgaggtggagaaccagccctggttcgcagccatctaccagaagaacaagggaggaagtcctccctcctttaaatgtggtgggagtctcatcagtccttgctgggtggccagtgccgcacactgcttcattcaactcccaaagaaggaaaactacgttgtctacctgggtcagtcgaaggagagctcctataatcctggagagatgaagtttgaggtggagcagctcatcttgcacgaatactacagggaagacagcctggcctaccataatgatattgccttgctgaagatacgtaccagcacgggccaatgtgcacagccatccaggtccatacagaccatctgcctgcccccaaggtttactgatgctccgtttggttcagactgtgagatcactggctttggaaaagagtctgaaagtgactatctctatccaaagaacctgaaaatgtccgtcgtaaagcttgtttctcatgaacagtgtatgcagccccactactatggctctgaaattaattataaaatgctgtgtgctgcggacccagagtggaaaacagattcctgcaagggcgattctggaggaccgcttatctgtaacatcgaaggccgcccaactctgagtgggattgtgagctggggccgaggatgtgcagagaaaaacaagcccggtgtctacacgagggtctcacacttcctggactggattcaatcccacattggagaagagaaaggtctggccttctga

SEQ ID NO: 11 - DNA, 639 bp, Homo sapiens
  source 1..639: mol_type = other DNA
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 12 - DNA, 636 bp, Mus musculus
  source 1..636: mol_type = other DNA
atgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctag

SEQ ID NO: 13 - DNA, 927 bp, Homo sapiens
  source 1..927: mol_type = other DNA
atgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatga

SEQ ID NO: 14 - DNA, 912 bp, Mus musculus
  source 1..912: mol_type = other DNA
atggccccgcccgcgctccaggcccagcctccaggcggctctcaactgaggttcctgctgttcctgctgctgttgctgctgctgctgtcatggccatcgcagggggacgccctggcaatgcctgaacagcgaccctccggccctgagtcccaactcaacgccgacgagctacggggtcgcttccaggacctgctgagccggctgcatgccaaccagagccgagaggactcgaactcagaaccaagtcctgacccagctgtccggatactcagtccagaggtgagattggggtcccacggccagctgctactccgcgtcaaccgggcgtcgctgagtcagggtctccccgaagcctaccgcgtgcaccgagcgctgctcctgctgacgccgacggcccgcccctgggacatcactaggcccctgaagcgtgcgctcagcctccggggaccccgtgctcccgcattacgcctgcgcctgacgccgcctccggacctggctatgctgccctctggcggcacgcagctggaactgcgcttacgggtagccgccggcagggggcgccgaagcgcgcatgcgcacccaagagactcgtgcccactgggtccggggcgctgctgtcacttggagactgtgcaggcaactcttgaagacttgggctggagcgactgggtgctgtccccgcgccagctgcagctgagcatgtgcgtgggcgagtgtccccacctgtatcgctccgcgaacacgcatgcgcagatcaaagcacgcctgcatggcctgcagcctgacaaggtgcctgccccgtgctgtgtcccctccagctacaccccggtggttcttatgcacaggacagacagtggtgtgtcactgcagacttatgatgacctggtggcccggggctgccactgcgcttga

SEQ ID NO: 15 - DNA, 2184 bp, Homo sapiens
  source 1..2184: mol_type = other DNA
atgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaaccgttctggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgttctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctctgcatcttctggaaggtcaattacaactttgaagatgagaccgtgaggaagtttctactgagccagttgcagaaaaccaggcctgtgatcttggacccagccgaacccacaggtgacgtgggtggaggggaccgttggtgttggcatcttctggcaaaagaagcaaaggaatggttatcctctccctgcttcaaggatgggactggaaacccaataccaccttggaaagtgccgacaatgcagacaccaggaagttgtggagctaggatccatcctattgtcaatgagatgttctcatccagaagccatagaatcctgaataataattctaaaagaaacttctggagatcatctggcaatcgcttttaa

SEQ ID NO: 16 - DNA, 447 bp, Mus musculus
  source 1..447: mol_type = other DNA
atgaagccaaaagcatctttacccccaaagtacgccctggagctgctcaccgtgtatgcctgggagcagggcagtggcacagatgactttgacattgctgaaggcttccggaccgtcctggacctggttataaaataccggcagctctgcatcttctggacagtcaattacaactttgaagaggaatacatgcggaagttcctactgacccagatccagaaaaagaggcctgtaatcctggatccagcagatcccacaggcgatgtgggaggaggtgaccgctggtgctggcatcttctagctgaagaagcgaaggagtggctgtcctccccttgtttccaagtggagcaaaaaggcctggtacagccttggaaagtgccagtaatgcagacccccggaagctgtggaggtcagatctaccccactgtgggtggagttactaagtag

SEQ ID NO: 17 - DNA, 300 bp, Homo sapiens
  source 1..300: mol_type = other DNA
atgaaagtctctgccgcccttctgtgcctgctgctcatagcagccaccttcattccccaagggctcgctcagccagatgcaatcaatgccccagtcacctgctgctataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttga

SEQ ID NO: 18 - DNA, 447 bp, Mus musculus
  source 1..447: mol_type = other DNA
atgcaggtccctgtcatgcttctgggcctgctgttcacagttgccggctggagcatccacgtgttggctcagccagatgcagttaacgccccactcacctgctgctactcattcaccagcaagatgatcccaatgagtaggctggagagctacaagaggatcaccagcagcaggtgtcccaaagaagctgtagtttttgtcaccaagctcaagagagaggtctgtgctgaccccaagaaggaatgggtccagacatacattaaaaacctggatcggaaccaaatgagatcagaacctacaactttatttaaaactgcatctgccctaaggtcttcagcacctttgaatgtgaagttgacccgtaaatctgaagctaatgcatccactaccttttccacaaccacctcaagcacttctgtaggagtgaccagtgtgacagtgaactag

SEQ ID NO: 19 - DNA, 1590 bp, Homo sapiens
  source 1..1590: mol_type = other DNA
atggagtggaaccctggcttccccttgagcatcgatgccaaatgccacaaggatttaccccgtgatatccagtttgatagtgaaaaaggagtggactttgttctgaattactccaaagcgatggagaacctgttcatcaaccgcttcatgcacatgttccagtcttcttggaatgacttcgccgactttgagaaaatctttgtcaagatcagcaacactatttctgagcgggtcatgaatcactggcaggaagacctgatgtttggctaccagttcctgaatggctgcaaccctgtgttgatccggcgctgcacagagctgcccgagaagctcccggtgaccacggagatggtagagtgcagcctggagcggcagctcagcttggagcaggaggtccagcaagggaacattttcatcgtggactttgagctgctggatggcatcgatgccaacaaaacagacccctgcacactccagttcctggccgctcccatctgcttgctgtataagaacctggccaacaagattgtccccattgccatccagctcaaccaaatcccgggagatgagaaccctattttcctcccttcggatgcaaaatacgactggcttttggccaaaatctgggtgcgttccagtgacttccacgtccaccagaccatcacccaccttctgcgaacacatctggtgtctgaggtttttggcattgcaatgtaccgccagctgcctgctgtgcaccccattttcaagctgctggtggcacacgtgagattcaccattgcaatcaacaccaaggcccgtgagcagctcatctgcgagtgtggcctctttgacaaggccaacgccacagggggcggtgggcacgtgcagatggtgcagagggccatgaaggacctgacctatgcctccctgtgctttcccgaggccatcaaggcccggggcatggagagcaaagaagacatcccctactacttctaccgggacgacgggctcctggtgtgggaagccatcaggacgttcacggccgaggtggtagacatctactacgagggcgaccaggtggtggaggaggacccggagctgcaggacttcgtgaacgatgtctacgtgtacggcatgcggggccgcaagtcctcaggcttccccaagtcggtcaagagccgggagcagctgtcggagtacctgaccgtggtgatcttcaccgcctccgcccagcacgccgcggtcaacttcggccagtacgactggtgctcctggatccccaatgcgcccccaaccatgcgagccccgccaccgactgccaagggcgtggtgaccattgagcagatcgtggacacgctgcccgaccgcggccgctcctgctggcatctgggtgcagtgtgggcgctgagccagttccaggaaaacgagctgttcctgggcatgtacccagaagagcattttatcgagaagcctgtgaaggaagccatggcccgattccgcaagaacctcgaggccattgtcagcgtgattgctgagcgcaacaagaagaagcagctgccatattactacttgtccccagaccggattccgaacagtgtggccatctga

SEQ ID NO: 20 - DNA, 2025 bp, Mus musculus
  source 1..2025: mol_type = other DNA
atgccctcctacacggtcaccgtggccaccggcagccagtggttcgcgggcaccgacgactacatctacctcagcctcattggctctgcgggctgtagcgagaagcatctgctggacaaggcattctacaatgacttcgaacggggcgcggtggactcctacgatgtcaccgtggatgaggagctgggcgagatctacctagtcaaaattgagaagcgcaaatactggctccatgatgactggtacctgaagtacatcacactgaagacaccccacggggactacatcgagttcccatgttaccgctggatcacaggcgagggcgagattgtcctgagggatggacgtgcaaaattggcccgagatgaccaaattcacatcctcaagcagcacagacgtaaagaactggaggcacggcaaaaacagtatcgatggatggagtggaaccccggcttccctttgagtattgatgccaaatgccacaaggatctgccccgagatatccagtttgatagtgaaaaaggagtggactttgttctgaactactcaaaagcgatggagaacctgttcatcaaccgcttcatgcacatgttccagtcttcctggcacgactttgctgactttgagaaaatcttcgtcaaaatcagcaacactatatctgagcgagtcaagaaccactggcaggaagacctcatgtttggctaccagttcctgaatggctgcaacccagtactcatcaagcgttgcacagcgttgcccccgaagctcccagtgaccacagagatggtggagtgcagcctagagcggcagctcagtttagaacaggaagtacaggaagggaacattttcatcgttgattacgagctactggatggcatcgatgctaacaaaactgacccctgtacacaccagttcctggctgcccccatctgcctgctatataagaacctagccaacaagattgttcccattgccatccagctcaaccaaacccctggagagagtaacccaatcttcctccctacggattcaaagtacgactggcttttggccaaaatctgggtgcgttccagtgacttccacgtccatcaaacgatcacccaccttctgcgcacgcatctggtgtctgaggtgtttggtatcgccatgtaccgccagctgcctgctgtgcatccccttttcaagctgctggtagcccatgtgaggttcaccattgctatcaacactaaggcccgggaacagcttatctgcgagtatggcctttttgacaaggccaatgccaccgggggtggagggcacgtgcagatggtgcagagggctgtccaggatctgacctattcctccctgtgtttcccggaggccatcaaggcccggggcatggacagcacggaagacatccccttctacttctatcgtgatgatggactgctcgtgtgggaggctatccagtcgttcacaatggaggtggtgagcatctactatgagaacgaccaggtggtggaggaggaccaggaactgcaggacttcgtgaaggatgtttacgtgtacggcatgcggggcaaaaaggcctcaggtttccccaagtccatcaagagcagggagaagctgtccgagtacctgacggtggtgatcttcacggcctctgcccagcatgcagctgtaaacttcggccagtatgactggtgctcctggatccccaacgctcctccaactatgcgggccccaccacccacggccaagggtgtggtcaccatcgagcagatcgtggatactctaccagaccgtggccgatcatgttggcatctaggtgcagtgtgggccttgagccagtttcaagaaaatgagctgtttctaggcatgtacccagaggagcatttcattgagaagccagtgaaggaagccatgatccgattccgcaagaacctggaggccatcatcagcgtgatcgccgagcgcaataagaacaaaaagctcccctactactacctgtcaccagacaggattcccaacagcgtagccatctaa

(19) United States
(12) Patent Application Publication
(10) Pub. No.: US 2025/0049773 A1
(43) Pub. Date: Feb. 13, 2025

(54) INHIBITION OF STEROL RESPONSE ELEMENT BINDING PROTEINS AS ATARGET FOR SELECTIVE ELIMINATION OF SENESCENT CELLS

(71) Applicant: Trustees of Tufts College, Medford, MA (US)

(72) Inventor: Christopher Wiley, Medford, MA (US)

(21) Appl. No.: 18/711,404

(22) PCT Filed: Nov. 23, 2022
(86) PCT No.: PCT/US2022/080434

§ 371 Date: May 17, 2024

Related U.S. Application Data
(60) Provisional application No. 63/264,510, filed on Nov. 23, 2021.

(51) Int. Cl. A61K 31/4439; A61K 31/047; A61K 31/12; A61K 31/704; A61P 43/00

(52) U.S. Cl. CPC A61K 31/4439; A61K 31/047; A61K 31/12; A61K 31/704; A61P 43/00

(57) ABSTRACT

Disclosed are methods of treating diseases or disorders associated with cellular senescence. The methods include administering an inhibitor of sterol response element binding proteins (SREBPs).

### CROSS-REFERENCE TO RELATED APPLICATIONS

The present application claims priority to U.S. Provisional Patent Application No. 63/264,510 that was filed Nov. 23, 2021, the entire contents of which are hereby incorporated by reference.

### STATEMENT REGARDING FEDERALLY SPONSORED RESEARCH OR DEVELOPMENT

This invention was made with government support under agreement 58-1950-4-003 awarded by the United States Department of Agriculture. The government has certain rights in the invention.

### SEQUENCE LISTING

A Sequence Listing accompanies this application and is submitted as an xml file of the sequence listing named “166118_01267.xml” which is 42,896 bytes in size and was created on Nov. 23, 2022. The sequence listing is electronically submitted via Patent Center and is incorporated herein by reference in its entirety.

### BACKGROUND

Senescent cells drive chronic degenerative conditions due to the secretion of a complex melange of biologically active molecules collectively known as the senescence-associated secretory phenotype, or SASP. Drugs that selectively eliminate senescent cells, or “senolytics” are in demand for the treatment of multiple age-related degenerative pathologies.

### SUMMARY OF THE INVENTION

Disclosed are methods for selecting eliminating senescent cells in a subject in need thereof. The methods include administering an inhibitor of sterol response element binding proteins (SREBPs).

In an aspect of the current disclosure, methods are provided. In some embodiments, the methods comprise administering a therapeutically effective amount of a sterol response element binding protein (SRE-BP) activation inhibitor to a subject in need thereof. In some embodiments, the subject is suffering from an age-related pathology. In some embodiments, the subject is suffering from a neurodegenerative disease. In some embodiments, the neurodegenerative disease is selected from multiple sclerosis (MS), Alzheimer's disease (AD), Parkinson's disease (PD), and amyotrophic lateral sclerosis (ALS). In some embodiments, the subject is suffering from Alzheimer's disease, Parkinson's disease, or Down syndrome. In some embodiments, the subject is suffering from a cell proliferative disease or disorder. In some embodiments, the cell proliferative disease or disorder is cancer. In some embodiments, the subject is being treated with a chemotherapeutic drug that induces senescence. In some embodiments, the chemotherapeutic drug that induces senescence is selected from the group consisting of doxorubicin, etoposide, and cisplatin. In some embodiments, the sterol response element binding protein (SRE-BP) activation inhibitor is a compound selected from xanthohumol, fatostatin, betulin, and PF-429242. In some embodiments, the compound is xanthohumol. In some embodiments, the therapeutically effective amount of the compound is about 60 mg/kg.

In another aspect of the current disclosure, methods of killing senescent cells in a subject in need thereof are provided. In some embodiments, the methods comprise administering a therapeutically effective amount of a sterol response element binding protein (SRE-BP) activation inhibitor in an amount sufficient to kill senescent cells in the subject. In some embodiments, the senescent cells are hepatocytes. In some embodiments, the subject is suffering from an age-related pathology. In some embodiments, the subject is suffering from a neurodegenerative disease. In some embodiments, the neurodegenerative disease is selected from multiple sclerosis (MS), Alzheimer's disease (AD), Parkinson's disease (PD), and amyotrophic lateral sclerosis (ALS). In some embodiments, the subject is suffering from Alzheimer's disease, Parkinson's disease, or Down syndrome. In some embodiments, the subject is suffering from a cell proliferative disease or disorder. In some embodiments, the cell proliferative disease or disorder is cancer. In some embodiments, the subject is being treated with a chemotherapeutic drug that induces senescence. In some embodiments, the chemotherapeutic drug that induces senescence is selected from the group consisting of doxorubicin, etoposide, and cisplatin. In some embodiments, the sterol response element binding protein (SRE-BP) activation inhibitor is a compound selected from xanthohumol, fatostatin, betulin, and PF-429242. In some embodiments, the compound is xanthohumol. In some embodiments, the therapeutically effective amount of the compound is about 60 mg/kg.

In another aspect of the current disclosure, methods of reducing a senescence-associated secretory phenotype (SASP) in cells of a subject in need thereof are provided. In some embodiments, the methods comprise administering a therapeutically effective amount of a sterol response element binding protein (SRE-BP) activation inhibitor to the subject to reduce the SASP in cells of the subject. In some embodiments, the cells of the subject are hepatocytes. In some embodiments, the subject is suffering from an age-related pathology. In some embodiments, the subject is suffering from a neurodegenerative disease. In some embodiments, the neurodegenerative disease is selected from multiple sclerosis (MS), Alzheimer's disease (AD), Parkinson's disease (PD), and amyotrophic lateral sclerosis (ALS). In some embodiments, the subject is suffering from Alzheimer's disease, Parkinson's disease, or Down syndrome. In some embodiments, the subject is suffering from a cell proliferative disease or disorder. In some embodiments, the cell proliferative disease or disorder is cancer. In some embodiments, the subject is being treated with a chemotherapeutic drug that induces senescence. In some embodiments, the chemotherapeutic drug that induces senescence is selected from the group consisting of doxorubicin, etoposide, and cisplatin. In some embodiments, the sterol response element binding protein (SRE-BP) activation inhibitor is a compound selected from xanthohumol, fatostatin, betulin, and PF-429242. In some embodiments, the compound is xanthohumol. In some embodiments, the therapeutically effective amount of the compound is about 60 mg/kg.

In another aspect of the current disclosure, methods of treating non-alcoholic fatty liver disease (NAFLD) in a subject in need thereof are provided. In some embodiments, the methods comprise administering a therapeutically effective amount of a sterol response element binding protein (SRE-BP) activation inhibitor to the subject to treat the NAFLD. In some embodiments, the SRE-BP activation inhibitor is selected from xanthohumol, fatostatin, betulin, and PF-429242.

In another aspect of the current disclosure, methods of treating a subject suffering from chemotherapy-induced cellular senescence are provided. In some embodiments, the methods comprise administering a therapeutically effective amount of a sterol response element binding protein (SRE-BP) activation inhibitor to the subject to treat the chemotherapy-induced cellular senescence. In some embodiments, the SRE-BP activation inhibitor is selected from xanthohumol, fatostatin, betulin, and PF-429242.

In another aspect of the current disclosure, kits, systems, and platforms are provided. In some embodiments, the kits, systems, or platforms comprise: reagents for detecting cellular senescence in a subject's cells; and one or more sterol response element binding protein (SRE-BP) activation inhibitor. In some embodiments, the one or more sterol response element binding protein (SRE-BP) activation inhibitor is selected from xanthohumol, fatostatin, betulin, and PF-429242. In some embodiments, the one or more sterol response element binding protein (SRE-BP) activation inhibitor is xanthohumol. In some embodiments, the reagents for detecting cellular senescence in a subject's cells comprise primers specific for detecting expression of one or more genes selected from matrix metalloproteinase 3 (MMP3), prostaglandin-endoperoxide synthase 2 (Ptgs2), serpin family E member 1 (Serpine1), encoding plasminogen activator inhibitor 1 (PAI-1), plasminogen activator, urokinase (Plau), interleukin 6 (11-6), growth differentiation factor 15 (Gdf15), 2′, 5′ oligoadenylate synthetase 2 (Oas2), C-C motif chemokine 2 (Ccl2), and arachidonate 5—In some embodiments, the reagents for detecting cellular senescence in a subject's cells comprise primers specific for detecting expression of matrix metalloproteinase 3 (MMP3), prostaglandin-endoperoxide synthase 2 (Ptgs2), serpin family E member 1 (Serpine1), encoding plasminogen activator inhibitor 1 (PAI-1), plasminogen activator, urokinase (Plau), interleukin 6 (11-6), growth differentiation factor 15 (Gdf15), 2′, 5′ oligoadenylate synthetase 2 (Oas2), C-C motif chemokine 2 (Ccl2), and arachidonate 5-lipoxygenase (Alox5) by quantitative reverse transcription polymerase chain reaction (qRT-PCR). In some embodiments, the reagents for detecting cellular senescence in a subject's cells further comprises a control sample.

### BRIEF DESCRIPTION OF THE FIGURES

FIGS. 1A and 1B. SRE-BP activation inhibitors selectively kill senescent cells. IMR-90 fibroblasts were induced to senesce with either 10 Gy of ionizing radiation (IR), 250 nM doxorubicin [SEN(DOXO)], or quiescent vehicle control (QUI) for 24 hours. Media was then replaced with drug-free growth media, and senescence was allowed to develop for 12 days. On day 12, growth media (10% serum) was replaced with quiescence media (0.2% serum+ITS supplement) for 48 hours to induce quiescence in non-senescent cells. On day 14, media was replaced with quiescence media containing increasing doses of fatostatin (A), xanthohumol (B), PF-429242 (C), or 30 micromolar betulin (D). DMSO served as a 0 micromolar vehicle control. \<0.001

FIGS. 2A, 2B, 2C, and 2D. The SRE-BP activation inhibitor xanthohumol lowers markers of senescence in vivo. Mice were fed a high fat diet (HFD) or high fat diet with 60 mg/kg xanthohumol (HFD+XN) for 12 weeks. RNA was extracted from mice and analyzed by quantitative PCR for (A) p21^{WAF1}, (B) p16^{INK4a}, (C) p15^{INK4b}, and (D) SASP factors.

### DETAILED DESCRIPTION

The present invention is described herein using several definitions, as set forth below and throughout the application.

Sterol response element binding proteins (SREBPs) are master regulators of lipid metabolism in most mammalian cells. In response to sterol (e.g. cholesterol) deficiency, these transcription factors become activated and are subject to cleavage reactions that allow them to translocate to the nucleus, where they activate transcription of enzymes involved in lipid synthesis. Senescent cells lose SREBP activity in a p53-dependent manner, this happens due to selective import of sterols (e.g. cholesterol) into the endoplasmic reticulum. These reticular sterols inhibit cholesterol and lipid synthesis in the cell, lowering total cholesterol by essentially tricking the cell into thinking it is replete with sterols.

This loss of SREBP activity is also a potential vulnerability of senescent cells. Since they have much lower levels of sterol and lipid synthesis, an inhibitor of SREBP activation could starve senescent cells of these lipids, resulting in cell death. Thus, inhibitors of SREBP activation are targets for senolytic drugs. As used herein, “senolytic” refers to the property of a molecule, compound, or composition to selectively kill senescent cells, whether by apoptosis or another form of cell death.

Therefore, the current disclosure provides methods for killing senescent cells in a subject in need thereof. As used herein, “cellular senescence”, first reported in 1961 by Hayflick and Moorehead, refers to a cell fate that entails essentially irreversible replicative arrest, sustained viability with resistance to apoptosis, and, frequently, increased or altered metabolic activity. Intra- and extracellular signals that can contribute to cells' adopting the senescent cell fate mainly include signals related to tissue or cellular damage and/or cancer development. These include DNA damage, telomeric uncapping or dysfunction, exposure to extracellular DNA, oncogene activation, replicative stress or inducers of proliferation (such as growth hormone/IGF-1), protein aggregates, misfolded proteins, failed protein removal through decreased autophagy, presence of advanced glycation endproducts (AGEs) due to the reaction of reducing sugars with amino groups in proteins (e.g. Haemoglobin A1c is an AGE), saturated lipids and other bioactive lipids (bradykines, certain prostaglandins, etc.), reactive metabolites (e.g. ROS, hypoxia or hyperoxia), mechanical stress (e.g. bone-on-bone stress in osteoarthritis or shear stress such as occurs on the venous side of AV fistulae for haemodialysis or around atherosclerotic plaques), inflammatory cytokines (e.g. TNFα), mitochondrial dysfunction (e.g. mitochondrial DNA depletion), damage-associated molecular patterns (DAMPs, e.g. released intracellular contents signalling breakage of neighbouring cells), and pathogen-associated molecular patterns (PAMPs, e.g. bacterial endotoxins). These inducers activate one or more senescence-promoting transcription factor cascades, in some cases involving p16^{INK4a}, retinoblastoma protein (Rb), in others, p53 and p21^{CIP1}, both of these pathways, or other pathways.

These transcription factor cascades enforce replicative arrest and cause altered expression of hundreds of genes as well as epigenetic changes in DNA. Cellular senescence can take longer to become established than other cell fates, such as replication, differentiation, apoptosis or necrosis. From initiation, the senescence program undergoes multiple permutations in gene expression and adopting alter secretory profiles over time, at least in cell culture, depending on the cell type and the inducers driving the cell into the senescent fate. Senescent cells also acquire a senescence-associated secretory phenotype (SASP). The SASP can include: 1) inflammatory, pro-apoptotic, insulin resistance-inducing cytokines, such as TNFα, interleukin- (IL-) 6, IL-8 and others, 2) chemokines that attract, activate and anchor immune cells, 3) matrix metalloproteinases (MMPs), such as MMP-3, -9 and -12 that cause tissue destruction, 4) TGFβ family members that can contribute to fibrosis and stem cell and progenitor dysfunction, 5) activins and inhibins that also induce stem cell and progenitor dysfunction and dysdifferentiation, 6) factors such as the serpines (e.g. plasminogen activator inhibitor [PAI]-1 and -2) that can cause blood clotting and fibrosis, 7) growth factors that can exacerbate tumour spread, 8) bioactive lipids that also contribute to inflammation and tissue dysfunction (e.g. bradykines, ceramides or prostaglandins), 9) micro-RNAs (miRNAs) that contribute to stem and progenitor cell dysfunction, inflammation and insulin resistance, and 10) exosomes that can carry cytotoxic and senescence-inducing cargos locally and systemically. See Kirkland and Tchkonia, J Intern Med. 2020 Aug. 4. In addition, senescent cells activate the biosynthesis of several oxylipins that promote segments of the SASP and reinforce the proliferative arrest. Notably, senescent cells synthesize and accumulate an unstudied intracellular prostaglandin, 1a,1b-dihomo-15-deoxy-delta-12,14-prostaglandin J2. Released 15-deoxy-delta-12,14-prostaglandin J2 is a biomarker of senolysis in culture and in vivo. This and other prostaglandin D2-related lipids promote the senescence arrest and SASP by activating RAS signaling. See Wiley et al. “Oxylipin biosynthesis reinforces cellular senescence and allows detection of senolysis”, Cell Metab. 2021 Jun. 1; 33(6):1124-1136, which is incorporated by reference herein.

Thus, methods of removing deleterious senescent cells, or “senolytics” are thought to have a therapeutic benefit. The inventor has discovered that senescent cells require signaling from sterol response element binding proteins (SREBPs) to maintain their survival. Thus, the inventor demonstrated that inhibitors of the activation of SREBPs are potent senolytic compounds. In some embodiments, the inhibitors of SREBP activation are fatostatin, betulin, xanthohumol, or PF-429242. Therefore, in some embodiments, the methods comprise administering an SRE-BP activation inhibitor to a subject in need thereof comprising a compound selected from the group consisting of fatostatin, betulin, xanthohumol, and PF429242 in an amount sufficient to treat the subject. In some embodiments, the methods comprise administering fatostatin to a subject in an amount sufficient to treat the subject. In some embodiments, the methods comprise administering betulin to a subject in an amount sufficient to treat the subject.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for a disease or disorder associated with cellular senescence. A subject in need thereof may include a subject suffering from an age-related disease or disorder. A subject in need thereof may include a subject having a cancer that is being treated by administering a therapeutic agent that induces cellular senescence. In some embodiments, a subject in need thereof is being treated with doxorubicin, etoposide, or cisplatin. A subject in need thereof may refer to a subject suffering from a neurodegenerative disease or disorder. In some embodiments, a subject in need thereof is suffering from Alzheimer's disease, Down syndrome, or Parkinson's disease.

The term “subject” may be used interchangeably with the terms “individual” and “patient” and includes human and non-human mammalian subjects.

The disclosed methods may be utilized to treat diseases and disorders associated with cellular senescence which may include, but are not limited to, age related diseases or disorders, neurodegenerative diseases or disorders, non-alcoholic fatty liver disease (NAFLD), or cancers being treated with chemotherapeutic drugs that induce cellular senescence. In some embodiments, the disclosed methods comprise administering fatostatin in an amount sufficient to treat the subject. In some embodiments, the disclosed methods comprise administering betulin in an amount sufficient to treat the subject. In some embodiments, the disclosed methods comprise administering xanthohumol in an amount sufficient to treat the subject, which may comprise about 60 mg/kg per dose. In some embodiments, the disclosed methods comprise administering PF-429242 in an amount sufficient to treat the subject.

In some embodiments, the subject is suffering from an age-related pathology. Though senescence plays physiological roles during normal development and is needed for tissue homeostasis, senescence constitutes a stress response triggered by insults associated with aging such as genomic instability and telomere attrition, which are primary aging hallmarks themselves. There is also an intimate link between senescence and the other antagonistic hallmarks of aging. For example, senescent cells display decreased mitophagy, resulting in an “old,” defective mitochondrial network that may contribute to metabolic dysfunction in age. See McHugh and Gil, Front. Cell. Neurosci., 11 Feb. 2020, which is incorporated herein by reference in its entirety.

In some embodiments, the subject is suffering from a neurodegenerative disease. The increased presence of senescent cells in different neurodegenerative diseases suggests the contribution of senescence in the pathophysiology of these disorders. Furthermore, there is an extensive body of literature that associates cellular senescence with several neurodegenerative disorders including Alzheimer's disease (AD), Down syndrome (DS), and Parkinson's disease (PD). See Martinez-Cue and Rueda. Front. Cell. Neurosci., 11 Feb. 2020, which is incorporated herein by reference in its entirety. As used herein, “Alzheimer's disease” refers to a progressive neurologic disorder that causes the brain to shrink (atrophy) and brain cells to die. Alzheimer's disease is the most common cause of dementia—a continuous decline in thinking, behavioral and social skills that affects a person's ability to function independently. As used herein, “Down syndrome” refers to a condition, also known as trisomy 21, caused by the presence of a third copy of chromosome 21 in a subject. As used herein, “Parkinson's disease” refers to a neurodegenerative disorder that affects predominately dopamine-producing (“dopaminergic”) neurons in a specific area of the brain called substantia nigra.

In some embodiments, the subject is suffering from cancer. Senescence is generally regarded as a tumor suppressive process, both by preventing cancer cell proliferation and suppressing malignant progression from pre-malignant to malignant disease. It may also be a key effector mechanism of many types of anticancer therapies, such as chemotherapy, radiotherapy, and endocrine therapies, both directly and via bioactive molecules released by senescent cells that may stimulate an immune response. However, senescence may contribute to reduced patient resilience to cancer therapies and may provide a pathway for disease recurrence after cancer therapy. See Wyld et al. Cancers. 2020 August; 12(8): 2134, which is incorporated herein by reference in its entirety. Therefore, drugs targeting senescent cells provide additional means to kill cells that are, for the moment, senescent but may re-activate and again become malignant. Thus, in some embodiments, the subject is being treated with a chemotherapeutic drug that induces senescence. Further, in some embodiments the drug is selected from the group consisting of doxorubicin, etoposide, bleomycin, and cisplatin.

### Chemical Entities.

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —R¹C(O)N(R²)—, —R¹C(O)N(R²) R³—, —C(O)NR²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

### Pharmaceutical Compositions:

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that kills senescent cells may be administered as a single compound or in combination with another compound that kills senescent cells or that has a different pharmacological activity, e.g., chemotherapeutic cells that induce senescence.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with cellular senescence. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with cellular senescence.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Methods of Using Inhibitors of SREBP Activation to Kill Senescent Cells:

Disclosed are methods of using the compounds and pharmaceutical compositions for treating a subject having or at risk for developing a disease or disorder associated with cellular senescence.

In some embodiments, the disclosed methods include treating a subject in need of treatment for a disease or disorder associated with cellular senescence by targeting sterol response element binding protein (SREBP) activation. In the disclosed methods, the subject may be administered an effective amount of a therapeutic agent that inhibits the activation of SREBP.

The disclosed methods may be performed in order to treat age-related diseases or disorders, neurodegenerative diseases or disorders, or to treat subjects for cancer with administration of compounds that induce cellular senescence. In addition, the disclosed methods may be used to treat non-alcoholic fatty liver disease (NAFLD).

Suitable therapeutic agents for use in the disclosed methods may include, but are not limited to, a compound having a formula of

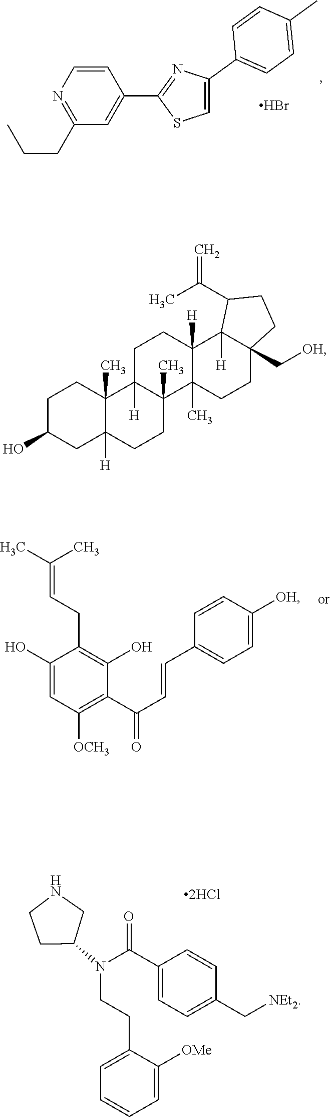

In some embodiments of the disclosed methods, the subject is administered a compound comprising fatostatin, betulin, xanthohumol, or PF-429242.

### Kits, Systems, and Platforms

The inventor has discovered that inhibitors of SRE-BP activation act as potent senolytics. Accordingly, in another aspect of the current disclosure, kits, systems, and platforms are provided. The kits, systems, and platforms may comprise reagents for detecting cellular senescence in a subject's cells; and one or more sterol response element binding protein (SRE-BP) activation inhibitor.

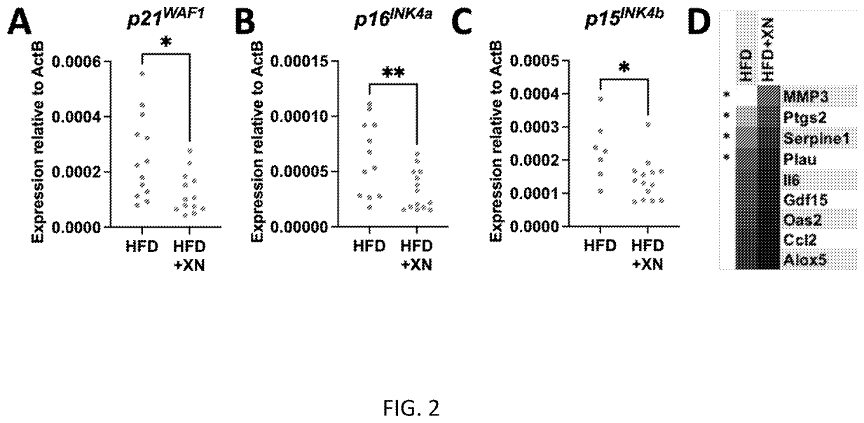

Referring now to FIG. 2D, the inventor has demonstrated that several genes characteristic of SASP are reduced in mouse livers after administration of an inhibitor of SRE-BP activation, i.e., xanthohumol, as measured by qRT-PCR. Therefore, the inventors contemplate that the kits, systems, and platforms may comprise reagents for detecting the expression of SASP genes, e.g., primers targeting the nucleotide sequences for the following genes: SEQ ID NOs:1-20, which correspond to the coding sequences for the genes displayed in FIG. 2D.

The design of appropriate primers to detect the nucleotide sequences SEQ ID NOs: 1-20 is within the ordinary skill in the art. In addition, SEQ ID NOs: 1, 3, 5, 7, 9, 11, 13, 15, 17, and 19 are human sequences for MMP3, PTGS2, SERPINE1, PAI-1, PLAU, IL-6, GDF15, OAS2, CCL2, and ALO5, respectively. Therefore, if the kits, systems, and platforms are intended to detect SASP in a human subject, primers designed to detect SEQ ID NOs: 1, 3, 5, 7, 9, 11, 13, 15, 17, and 19 are used. Nucleotide sequences for mouse Mmp3, Ptgs2, Serpine1, Pai-1, Plau, 116, Gdf15, Oas2, Ccl2, and Alox5 are SEQ ID NOs: 2, 4, 6, 8, 10, 12, 14, 16, 18, and 20, respectively. Furthermore, detection of SASP in cells of a subject may comprise obtaining an appropriate sample from the subject. For example, an appropriate sample may comprise a saliva sample, a blood sample, a urine sample, a fecal sample, or a biopsy such as a liver biopsy. Processing such samples to extract RNA are considered routine in the art.

### EXEMPLARY EMBODIMENTS

1. A method of killing senescent cells in a subject in need thereof, the method comprising administering fatostatin in an amount sufficient to treat the subject.
2. The method of embodiment 1, wherein the subject is suffering from an age-related pathology.
3. The method of embodiments 1 or 2, wherein the subject is suffering from a neurodegenerative disease.
4. The method of any of embodiments 1-3, wherein the subject is suffering from Alzheimer's disease, Parkinson's disease, or Down syndrome.
5. The method of embodiment 1, wherein the subject is suffering from cancer.
6. The method of embodiment 5, wherein the subject is being treated with a chemotherapeutic drug that induces senescence.
7. The method of embodiment 6, wherein the drug is selected from the group consisting of doxorubicin, etoposide, and cisplatin.
8. A method of killing senescent cells in a subject in need thereof, the method comprising administering betulin in an amount sufficient to treat the subject.
9. The method of embodiment 8, wherein the subject is suffering from an age-related pathology.
10. The method of embodiments 8 or 9, wherein the subject is suffering from a neurodegenerative disease.
11. The method of any of embodiments 8-10, wherein the subject is suffering from Alzheimer's disease, Parkinson's disease, or Down syndrome.
12. The method of embodiment 8, wherein the subject is suffering from cancer.
13. The method of embodiment 12, wherein the subject is being treated with a chemotherapeutic drug that induces senescence.
14. The method of embodiment 13, wherein the drug is selected from the group consisting of doxorubicin, etoposide, and cisplatin.

### EXAMPLES

The following Examples are illustrative and should not be interpreted to limit the scope of the claimed subject matter.

### Example 1—Inhibitors of SRE-BP Activation are Selectively Senolytic

### Results

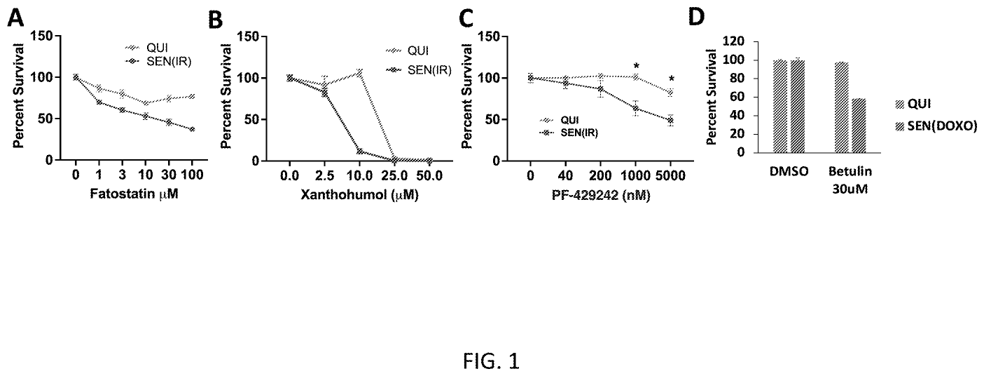

Incubation of cells with fatostatin resulted in increased cell death in senescent cells compared to quiescent cells (Figure TA). In particular, 3, 30, and 100 μM fatostatin was significantly more toxic to senescent cells than quiescent cells, though it is believed that 10 μM fatostatin is also toxic, but rather may require further replicates to achieve statistical significance. Furthermore, incubation of cells with 30 μM betulin was significantly more toxic to senescent cells than quiescent cells (FIG. 1B).

### Example 2—Treatment of Age-Related Diseases or Disorders with Inhibitors of SREBP Activation

As an organism ages, it accumulates genotoxic and other stresses that lead to an increase in senescent cells. Without being bound by any theory or mechanism, senescent cells are believed to produce factors that exacerbate or even underly negative health consequences associated with aging. Therefore, senolytic drugs may be used to treat subjects suffering from an age-related disease or disorder. In some embodiments of the current disclosure, fatostatin, xanthohumol or betulin may be administered to a subject suffering from an age-related disease or disorder. In some embodiments, a combination of two or more of fatostatin, xanthohumol, and betulin may be administered to the subject. Administration of fatostatin, xanthohumol, or betulin may be performed by any route suitable for administration of the compounds including, but not limited to oral, transdermal, percutaneous, intravenous, intramuscular, intranasal, buccal, intrathecal, intracerebral, or intrarectal routes. Treatment may comprise administration of an amount of fatostatin, xanthohumol, or betulin sufficient to treat the age-related disease or disorder. Thus, administration of fatostatin, xanthohumol, or betulin to the subject may cause the senescent cells to die, alleviating the signs and symptoms of the age-related disease or disorder.

### Example 3—Treatment of Neurodegenerative Diseases with Inhibitors of SREBP Activation

Neurodegenerative diseases have been associated with increased presence of senescent cells. Without being bound by any theory or mechanism, senescent cells are believed to play a role in neurodegeneration. Therefore, senolytic drugs may be used to treat subjects suffering from a neurodegenerative disorder, for example, Alzheimer's disease (AD), Parkinson's disease (PD), or Down syndrome (DS). In some embodiments of the current disclosure, fatostatin, xanthohumol, or betulin may be administered to a subject suffering from a neurodegenerative disease. In some embodiments, some combination of fatostatin, xanthohumol and betulin may be administered to the subject. Administration of fatostatin, xanthohumol, or betulin may be performed by any route suitable for administration of the compounds including, but not limited to oral, transdermal, percutaneous, intravenous, intramuscular, intranasal, buccal, intrathecal, intracerebral, or intrarectal routes. Treatment may comprise administration of an amount of fatostatin, xanthohumol, or betulin sufficient to treat the neurodegenerative disease. Thus, administration of fatostatin, xanthohumol, or betulin to the subject may cause the senescent cells to die, alleviating the signs and symptoms of the neurodegenerative disease.

### Example 4—Treatment of Cancer with Inhibitors of SREBP Activation

Senescence is a key mechanism of tumor suppression. This may be mediated by the DNA damage response (DDR) or by key oncogenes. Chemotherapy may cause cell death, often by apoptosis, resulting clinically in tumor regression. It may also cause cellular senescence, leading clinically to tumor stasis (growth arrest). Many types of chemotherapy cause DNA damage (DNA strand breaks or cross linking), which can, if severe, cause cell death via the DNA damage response (DDR), or they may trigger a non-lethal DDR, leading to acute or chronic senescence, depending on the extent and duration of the stimulus. Entry into senescence or cell death may also depend on whether the cell has functional tumor suppressor genes, such as p53 or p16^{INK4A} to regulate cell behavior. Different types of chemotherapy damage DNA in distinct ways. For example, doxorubicin prevents the resealing of the DNA double helix by inhibiting topoisomerase 2, which triggers a DDR and thereby may cause senescence. Others, such as vinca alkaloids, e.g., vinblastine (VBL), vinorelbine (VRL), vincristine (VCR) and vindesine (VDS), and taxanes, e.g., taxol, work by causing damage to the mitotic spindle during mitosis, resulting in cell death. Cyclophosphamide causes DNA cross linking, which again may trigger a DDR. There have been concerns that these senescent cells may be resistant to further damage from chemotherapy and be a potential reservoir for recurrence. There is evidence that senescent cells may also be re-programmed to re-enter the cell cycle after certain types of chemotherapy and may acquire a more stem cell-like phenotype, which may in turn contribute to tumor regrowth and evolution. See Wyld et al., supra. Thus, cancer may be treated with an agent that kills senescent cells. Moreover, senescent cells also promote the deleterious side effects of many chemotherapeutics and may promote cancer recurrence and metastasis. See, for example, Demaria M. et al. “Cellular Senescence Promotes Adverse Effects of Chemotherapy and Cancer Relapse”, Cancer Discov. 2017 February; 7(2):165-176; and Alimirah F. et al. “Cellular Senescence Promotes Skin Carcinogenesis through p38MAPK and p44/42MAPK Signaling”, Cancer Res. 2020 Sep. 1; 80(17):3606-3619, which are both incorporated by reference herein.

In some embodiments of the current disclosure, fatostatin, xanthohumol, or betulin may be administered to a subject suffering from a cell proliferative disease or disorder, for example, cancer. Administration of fatostatin, xanthohumol, or betulin may be performed by any route suitable for administration of the compounds including, but not limited to oral, transdermal, percutaneous, intravenous, intramuscular, intranasal, buccal, intrathecal, intracerebral, or intrarectal routes. Treatment may comprise administration of an amount of fatostatin or betulin sufficient to treat the cancer. Thus, administration of fatostatin, xanthohumol. or betulin to the subject may cause the senescent cells to die, alleviating, at least partially, the signs and symptoms of the cancer. In some embodiments, fatostatin, xanthohumol, or betulin may be administered at substantially the same time as the chemotherapeutic drug. In some embodiments, fatostatin, xanthohumol, or betulin may be administered before or after the chemotherapeutic drug. In some embodiments, some combination of fatostatin, xanthohumol, and/or betulin may be administered to the subject. In some embodiments, the administration of fatostatin, xanthohumol, or betulin may result in reduction in the size of a tumor. In some embodiments, the administration of fatostatin, xanthohumol, or betulin may result in prevention of recurrence of a tumor. The administration of fatostatin, xanthohumol, or betulin may also result in reduction in prevalence of malignant cells in the bloodstream or lymphatic system in subjects suffering from a hematological malignancy.

### Example 5—SRE-BP Activation Inhibitor Lowers Markers of Senescence In Vivo

Prolonged high fat diet (HFD) induces the generation of senescent cells in the liver. To test the hypothesis that inhibitors of SRE-BP activation are senolytic, two groups of mice were fed a HFD for 12 weeks. A control group was fed a HFD alone (FIG. 2, HFD), and an experimental group (FIG. 2, HFD+XN) was fed a HFD and the SRE-BP activation inhibitor xanthohumol (XN). RNA was isolated from liver samples from each group and subjected to quantitative reverse transcriptase polymerase chain reaction (qRT-PCR). FIGS. 2A, 2B, and 2C demonstrate that the HFD+XN group had reduced expression of the markers of senescence p21^{WAF1}, p16^{INK4a} and p15^{INK4b}, as compared to the control group, supporting the hypothesis that SRE-BP activation inhibitors, e.g., xanthohumol, act as senolytics. FIG. 2D demonstrates that the HFD+XN group has reduced expression of matrix metalloproteinase 3 (MMP3), prostaglandin-endoperoxide synthase 2 (Ptgs2), serpin family E member 1 (Serpine1), encoding plasminogen activator inhibitor 1 (PAI-1), plasminogen activator, urokinase (Plau), interleukin 6 (IL-6), growth differentiation factor 15 (Gdf15), 2′, 5′ oligoadenylate synthetase 2 (Oas2), C-C motif chemokine 2 (Ccl2), and arachidonate 5-lipoxygenase (Alox5), expression of which is characteristic of a senescence-associated secretory phenotype (SASP). Accordingly, the data presented in FIG. 2D supports the hypothesis that SRE-BP activation inhibitors, e.g., xanthohumol, are senolytic.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. (canceled)

2. A method of killing senescent cells in a subject in need thereof, the method comprising administering a therapeutically effective amount of a sterol response element binding protein (SRE-BP) activation inhibitor in an amount sufficient to kill senescent cells in the subject.

3. The method of claim 2, wherein the method reduces a senescence-associated secretory phenotype (SASP) in cells of the subject.

4. The method of claim 3, wherein the cells of the subject are hepatocytes.

5. The method of claim 2, wherein the subject is suffering from an age-related pathology.

6. The method of claim 2, wherein the subject is suffering from a neurodegenerative disease.

7. The method of claim 6, wherein the neurodegenerative disease is selected from multiple sclerosis (MS), Alzheimer's disease (AD), Parkinson's disease (PD), and amyotrophic lateral sclerosis (ALS).

8. The method of claim 2, wherein the subject is suffering from Alzheimer's disease, Parkinson's disease, or Down syndrome.

9. The method of claim 2, wherein the subject is suffering from a cell proliferative disease or disorder.

10. The method of claim 9, wherein the cell proliferative disease or disorder is cancer.

11. The method of claim 10, wherein the subject is being treated with a chemotherapeutic drug that induces senescence.

12. The method of claim 11, wherein the chemotherapeutic drug that induces senescence is selected from the group consisting of doxorubicin, etoposide, and cisplatin.

13. The method of claim 2, wherein the sterol response element binding protein (SRE-BP) activation inhibitor is a compound selected from xanthohumol, fatostatin, betulin, and PF-429242.

14. The method of claim 13, wherein the compound is xanthohumol.

15. The method of claim 14, wherein the therapeutically effective amount is about 60 mg/kg.

16. A method of treating non-alcoholic fatty liver disease (NAFLD) in a subject in need thereof, the method comprising administering a therapeutically effective amount of a sterol response element binding protein (SRE-BP) activation inhibitor to the subject to treat the NAFLD.

17. The method of claim 16, wherein the SRE-BP activation inhibitor is selected from xanthohumol, fatostatin, betulin, and PF-429242.

18-19. (canceled)

20. A kit comprising:
a. reagents for detecting cellular senescence in a subject's cells; and
b. one or more sterol response element binding protein (SRE-BP) activation inhibitor.

21. The kit of claim 20, wherein the one or more sterol response element binding protein (SRE-BP) activation inhibitor is selected from xanthohumol, fatostatin, betulin, and PF-429242.

22. The kit of claim 21, wherein the one or more sterol response element binding protein (SRE-BP) activation inhibitor is xanthohumol.

23. The kit of claim 20, wherein the reagents for detecting cellular senescence in a subject's cells comprise primers specific for detecting expression of one or more genes selected from matrix metalloproteinase 3 (MMP3), prostaglandin-endoperoxide synthase 2 (Ptgs2), serpin family E member 1 (Serpine1), encoding plasminogen activator inhibitor 1 (PAI-1), plasminogen activator, urokinase (Plau), interleukin 6 (Il-6), growth differentiation factor 15 (Gdf15), 2′, 5′ oligoadenylate synthetase 2 (Oas2), C-C motif chemokine 2 (Ccl2), and arachidonate 5-lipoxygenase (Alox5) by quantitative reverse transcription polymerase chain reaction (qRT-PCR).

24-25. (canceled)
